(19) United States
(12) Patent Application Publication
(10) Pub. No.: US 2024/0123071 A1
(43) Pub. Date: Apr. 18, 2024

(54) PREPARATION METHOD AND APPLICATION OF CTL CELL

(71) Applicant: Guangzhou Anjie Biomedical Technology CO., LTD., Guangdong (CN)

(72) Inventors: Chao Zhou, Guangdong (CN); Hong An, Guangdong (CN); Youde Lu, Guangdong (CN); Ling Zhou, Guangdong (CN); Yongbiao Du, Guangdong (CN); Jiaqi Tu, Guangdong (CN); Haibin Michael Yin, Guangdong (CN)

(21) Appl. No.: 17/621,687

(22) PCT Filed: May 22, 2020
(86) PCT No.: PCT/CN2020/091781

§ 371 Date: Dec. 21, 2021

(30) Foreign Application Priority Data
Jun. 24, 2019 (CN) 2019105475423

(51) Int. Cl. A61K 39/00; C07K 14/535; C12N 5/0783; C12N 9/16; C12N 9/22; C12N 15/11; C12N 15/90

(52) U.S. Cl. CPC A61K 39/464493; A61K 39/001193; A61K 39/4636; C07K 14/535; C12N 5/0638; C12N 9/16; C12N 9/22; C12N 15/11; C12N 15/907; C12Y 301/03002; A61K 2039/884; A61K 2239/58; C07K 2319/02; C12N 2310/20; C12N 2501/22; C12N 2800/80

(57) ABSTRACT

Provided are a preparation method for a CTL cell and an application thereof. The preparation method comprises the following steps of: inducing a CTL cell by using a tumor antigen PAP-GM-CSF sensitized DC cell; and knocking out a PD-1 gene of the CTL cell to obtain a PD-1 knock-out CTL cell. The CTL cell obtained by the preparation method can be used for preparing drugs for treatment of prostate cancer, especially for treating PAP-positive prostate cancer. The CTL cell does not cause CTL cell failure and anergy due to the tumor-expressed PD-L1 after being transfused into the body, thereby producing efficient specific cytotoxic effect on a tumor cell and improving the curative effect and reducing the side effect.

### CROSS-REFERENCE TO RELATED APPLICATIONS

The present application is a national phase entry under 35 USC § 371 of International Application PCT/CN2020/091781, filed May 22, 2020, which claims the benefit of and priority to Chinese Patent Application No. 201910547542.3, filed Jun. 24, 2019, the entire disclosures of which are incorporated by reference herein.

### SEQUENCE LISTING

This application contains a Sequence Listing, which was submitted in ASCII format via EFS-Web, and is hereby incorporated by reference in its entirety. The ASCII copy, created on 23 Sep. 2022, is named “2234-11-PCT-US-Sequence-Listing-09-23-2022” and is 27,188 bytes in size.

### TECHNICAL FIELD

The present disclosure belongs to the field of biotechnologies, and more particularly, relates to a preparation method for a CTL cell and an application thereof.

### BACKGROUND

Prostate cancer (PC) is one of the most common malignant tumors in a male genitourinary system, and is a local organ canceration disease, and a course of the disease can be divided into two stages, which are a hormone-dependent stage and a hormone-independent stage. In the hormone-dependent stage, prostatectomy, radiotherapy and medical castration can be used for treatment, accompanied by serious toxic and side effects, thereby affecting the quality of life of patients. After the hormone-dependent stage, the disease may progress to the hormone-independent stage, and almost all patients will suffer from castration resistant prostate cancer (CRPC), while metastatic castration resistant prostate cancer (mCRPC) is a main lethal factor of the prostate cancer. An average median life time of patients suffering from mCRPC is less than 2 years. Therefore, it is urgent to develop a new and effective therapy for the prostate cancer.

Cell therapy, as a new means of tumor treatment, has become a research hotspot, and especially, the treatment of the prostate cancer based on a dendritic cell (DC) vaccine has achieved good clinical results. Sensitizing the DC with a prostate cancer-related antigen peptide can induce a strong anti-tumor immune effect, however, there is a problem that the infused DC cell and the tumor antigen-specific CTL induced by the DC are inhibited by the tumor immunosuppressive micro-environment, leading to functional failure and anergy. The tumor micro-environment is a difficult problem in the treatment of a solid tumor, the tumor micro-environment which is composed of immune checkpoint mediated immunosuppressive signals (such as PD-1, CTLA-4, LAG-3 and TIM-3) plays an important role in promoting tumor immune escape. Therefore, the combination of a tumor vaccine and an immune checkpoint monoclonal antibody for treating the prostate cancer has become an attractive strategy. The prostate cancer is treated by combining Sipuleucel-T with an immune checkpoint inhibitor in a plurality of clinical trials registered in clinicaltrials.gov (such as Sipuleucel-T and Ipilimumab for Advanced Prostate Cancer (NCT01832870), A Randomized Phase 2 Trial of Combining Sipuleucel-T With Immediate vs. Delayed CTLA-4 Blockade for Prostate Cancer (NCT01804465)). In addition, there are many clinical trials for treating the prostate cancer by combining a DNA vaccine with the immune checkpoint inhibitor (NCT03600350, NCT02499835, NCT03815942, etc.). However, the long-term use of the immune checkpoint inhibitor may destroy immune tolerance, leading to serious side effects.

### SUMMARY

The present disclosure aims to edit a gene of a tumor antigen-specific CTL, knock out an immune checkpoint gene PD-1, and obtain a more powerful CTL cell unresponsive to an immunosuppressive micro-environment of a solid tumor.

The technical solutions used in the present disclosure are as follows.

A preparation method for a CTL cell comprises the following steps of: inducing a CTL cell by using a DC cell sensitized with a tumor antigen PAP-GM-CSF; and knocking out PD-1 gene of the CTL cell to obtain a PD-1 knock-out CTL cell.

Further, the tumor antigen PAP-GM-CSF is composed of PAP and GM-CSF linked by two amino acids Gly-Ser.

Further, the PAP upstream of the tumor antigen PAP-GM-CSF comprises a signal peptide.

Further, the nucleotide of the tumor antigen PAP-GM-CSF is shown in SEQ ID NO.: 1.

Further, the amino acid of the tumor antigen PAP-GM-CSF is shown in SEQ ID NO.: 2.

Further, the tumor antigen PAP-GM-CSF is expressed by a genetic engineering method.

Further, the genetic engineering method is selected from one of insect cell baculovirus expression system, HEK293 cell expression system, yeast expression system and Escherichia coli expression system; and preferably, the genetic engineering method is insect cell baculovirus expression system.

Further, the tumor antigen PAP-GM-CSF is obtained by purification after being expressed by the genetic engineering method.

Further, the purity of the tumor antigen PAP-GM-CSF is no less than 98%.

Further, the purification comprises ultrafiltration and continuous column chromatography.

Further, the continuous column chromatography comprises at least one selected from the group consisting of ion exchange, hydrophobic chromatography, hydroxyapatite chromatography and affinity chromatography.

Further, the continuous column chromatography is at least one selected from the group consisting of cation column EMD SO₃⁻ (M) flow-through, anion column EMD TMAE(M) and hydrophobic column Capto Butyl.

Further, a preparation method for the tumor antigen PAP-GM-CSF comprises the following steps of:
(1) constructing a shuttle plasmid pFast-Bac1-PAP-GM-CSF with pFast-Bac1 as a skeleton vector;
(2) transforming the shuttle plasmid into an Escherichia coli competent cell, and screening to obtain a recombinant bacmid PAP-GM-CSF-Bacmid;
(3) transfecting the recombinant bacmid into an insect cell, and after the cell has an obvious pathological change, acquiring the supernatant which is the first-generation baculovirus;
(4) infecting the insect cell with the first-generation baculovirus, and collecting the second-generation baculovirus or the third-generation baculovirus; and
(5) expressing PAP-GM-CSF by using a suspended insect cell infected and acclimated with the second-generation baculovirus or the-third generation baculovirus.

Further, a preparation method for the tumor antigen PAP-GM-CSF comprises the following steps of:
(1) constructing a shuttle plasmid pFast-Bac1-PAP-GM-CSF with pFast-Bac1 as a skeleton vector;
(2) transforming the shuttle plasmid into an Escherichia coli competent cell DH10bac, and screening to obtain a recombinant bacmid PAP-GM-CSF-Bacmid;
(3) transfecting the recombinant bacmid into an insect cell Sf-9, and after the cell has an obvious pathological change, acquiring the supernatant which is the first-generation baculovirus;
(4) infecting the insect cell Sf-9 with the first-generation baculovirus, and collecting the second-generation baculovirus or the third-generation baculovirus; and
(5) expressing PAP-GM-CSF fusion protein by using a suspended insect cell Sf-9 infected and acclimated with the second-generation baculovirus or the third-generation baculovirus.

Further, the DC cell is selected from a human peripheral blood mononuclear cell (PBMC), a human peripheral blood CD14⁺ cell or bone marrow.

Further, sensitizing the DC cell with the tumor antigen PAP-GM-CSF comprises the following steps of: adding the DC cell into a lymphocyte serum-free medium containing rhGM-CSF and rhIL-4; and after culturing, adding the tumor antigen PAP-GM-CSF and TNF-β for induction to obtain the DC cell sensitized with the tumor antigen PAP-GM-CSF.

Further, sensitizing the DC cell with the tumor antigen PAP-GM-CSF comprises the following steps of: separating a peripheral blood mononuclear cell (PBMC) by a blood cell separator or Ficoll, separating a CD14⁺ monocyte by a magnetic bead sorting method, adding an X-VIVO™15 (LONZA) lymphocyte serum-free medium (containing 500 U/mL to 1000 U/mL rhGM-CSF and 500 U/mL rhIL-4), culturing in a CO₂ incubator at 37° C. under 5% CO₂, adding the PAP-GM-CSF fusion protein on the 5th day to stimulate activation of DC, adding 20 ng/mL TNF-β at the same time to induce maturation of DC, and culturing continuously for 48 hours.

Further, inducing the CTL cell by using the DC cell sensitized with the tumor antigen PAP-GM-CSF comprises the following steps of: acquiring a human peripheral blood mononuclear cell with the same source as the DC cell, and adding the human peripheral blood mononuclear cell into the DC cell sensitized with the tumor antigen PAP-GM-CSF for co-culture to induce the CTL cell.

Further, inducing the CTL cell by using the DC cell sensitized with the tumor antigen PAP-GM-CSF comprises the following steps of: separating the PBMC with the same source as the DC cell by a blood cell separator or Ficoll, adding the PBMC into the mature DC stimulated with PAP-GM-CSF (DC: PBMC=1: 10), and co-incubating in an incubator at 37° C. under 5% CO₂ for 3 days to 5 days.

Further, at least one selected from the group consisting of CRISPR/Cas9 system, TALEN system and zinc finger nuclease system are used to knock out PD-1 gene of the CTL cell; and preferably, CRISPR/Cas9 system is used to knock out PD-1 gene of the CTL cell.

Further, CRISPR/Cas9 system is used to knock out PD-1 gene of the CTL, which comprises the following step of: co-transfecting a Cas9 nuclease element and a gRNA targeting PD-1 gene into the CTL cell; and preferably, the co-transfection is implemented by electroporation transfection.

Further, the Cas9 nuclease element is a plasmid, a mRNA or a protein.

Further, the gRNA targeting PD-1 gene is composed of a targeted RNA and a guide RNA scaffold.

Further, the nucleotide sequence of the targeted RNA is shown in SEQ ID NO: 3 to 110, and the nucleotide sequence of the guide RNA scaffold is shown in SEQ ID NO: 111.

The present disclosure further provides a kit for acquiring a CTL cell, wherein the kit comprises the above-mentioned tumor antigen PAP-GM-CSF; and preferably, the kit further comprises the above-mentioned Cas9 nuclease element, the above-mentioned gRNA targeting PD-1 gene, and a kit instruction, and the above-mentioned preparation methods are recorded in the kit instruction.

The present disclosure further provides a use of the above-mentioned preparation method for the CTL cell in preparing drugs for treating prostate cancer.

The present disclosure further provides an use of the above-mentioned kit in preparing drugs for treating prostate cancer.

The present disclosure further provides an use of the above-mentioned preparation method for the CTL cell in preparing drugs for treating PAP-positive prostate cancer.

The present disclosure further provides an use of the above-mentioned kit in preparing drugs for treating PAP-positive prostate cancer.

The present disclosure has the beneficial effects as follows.

1. The present disclosure provides a preparation method for the PD-1 knock-out CTL cell, and the DC cell is sensitized with the tumor antigen PAP-GM-CSF; about 95% of prostate cancer express prostate acid phosphatase (PAP), the expression of which is mainly limited to prostate tissue, and PAP is widely expressed in patients with prostate cancer and has a good specificity, therefore PAP can be used as a target of a therapeutic prostate cancer vaccine. However, the PAP can only mediate the CD4⁺ cell to produce humoral immunity through a MHC II exogenous pathway, and induce a plasmocyte to produce an antibody, but cannot mediate the CD8⁺ cell (CTL cell) to produce cytotoxic effect through a MHC I endogenous pathway. However, a compound composed of PAP and GM-CSF as the tumor antigen can mediate the CD8⁺ cell (CTL cell) to produce the cytotoxic effect through the MHC I endogenous pathway.

2. The present disclosure provides a preparation method for the PD-1 knock-out CTL cell, the tumor antigen PAP-GM-CSF has a purity no less than 98%, and its immunogenicity is confirmed by mice immunization tests. In addition, the ingredient is definite and single, and can be large-scale produced after expanding the purification process, with a high stability between batches.

3. The present disclosure provides a preparation method for the PD-1 knock-out CTL cell, the PD-1 knock-out CTL cell does not cause CTL failure and anergy due to the tumor-expressed PD-L1 after being transfused into the body, thereby producing an efficient specific cytotoxic effect on a tumor cell and improving the curative effect.

### BRIEF DESCRIPTION OF THE DRAWINGS

The accompanying drawings described herein are used for providing further understanding for the present disclosure, and constitute a part of this application. Exemplary embodiments of the present disclosure and the descriptions thereof are used for explaining the present disclosure, and do not constitute any inappropriate limitation to the present disclosure. In the drawings:

FIG. 1 shows identification of a recombinant bacmid PAP-GM-CSF-Bacmid;

FIG. 2 shows expression of PAP-GM-CSF of a P2-generation baculovirus D detected by Western blotting;

FIG. 3 shows a purified PAP-GM-CSF protein detected by SDS-PAGE;

FIG. 4 shows a purified PAP-GM-CSF protein analyzed by HPLC;

FIG. 5 shows a phenotype of a sensitized DC cell detected by flow cytometry;

FIG. 6 shows a proportion of CTL cells induced by the DC detected by flow cytometry;

FIG. 7 shows expression of PD-1 of the CTL cells detected by flow cytometry; and

FIG. 8 shows knockout of the PD-1 detected by sanger sequencing.

### DETAILED DESCRIPTION

The technical solutions of the present disclosure are further described hereinafter with reference to the accompanying drawings and the specific embodiments, but the present disclosure is not limited to the specific embodiments. Unless otherwise specified, the materials, reagents, and the like used in the embodiments may all be obtained commercially.

Programmed death 1 (PD-1) belongs to a member of an immunoglobulin superfamily, and is an important immunosuppressive molecule. PD-1 has two ligands, i.e. PD-L1 and PD-L2, and PD-1 interacted with the ligands to transmit the inhibitory signal, and exerts a negative regulatory effect in immune response.

Cytotoxic T lymphocyte (CTL) is a subdivision of white blood cell, is a specific T cell, specially secretes various cytokines to participate in immunization, and has a killing effect on some antigens such as viruses and tumor cells.

Prostatic acid phosphatase (PAP) is a glycoprotein, is an isozyme of an acid phosphatase, is produced by lysosomes of prostate epithelial cells, and has a low serum content under normal circumstances, and the serum PAP will be increased in different degrees when blood-prostate barrier is destroyed by prostate lesion.

Granulocyte-macrophage colony stimulating factor (GM-CSF) is a white blood cell growth factor, can stimulate colony formation of neutrophils and macrophages in vitro, and has a function of promoting proliferation and development of megakaryocyte-erythroid progenitors and eosinophil precursors.

Dendritic cell (DC) is the most powerful antigen presenting cell (APC) in the body, and can efficiently uptake, process and present antigens. An immature DC has a strong migration ability, and a mature DC can effectively activate initial T cells, and is in the central link of starting, regulating and maintaining immune response.

Glycine (Gly) is a non-essential amino acid of human body, has both acidic and basic functional groups in molecules, may be ionized in water, has a strong hydrophilicity, belongs to the polar amino acid, is soluble in a polar solvent, but is hardly soluble in a nonpolar solvent, and has high boiling point and melting point.

Serine (Ser) is a non-essential amino acid of human body, and plays a role in muscle growth and metabolism of fat and fatty acids.

Interleukin-4 (IL-4) is a cytokine produced by various cells and acting on various cells, can stimulate proliferation of activated B cells and T cells, and plays a key role in regulating humoral immunity and adaptive immunity.

Tumor necrosis factor-α (TNF-α) is a cytokine capable of directly killing tumor cells without obvious toxicity to normal cells, and is one of bioactive factors with the strongest directly killing effect on tumors discovered so far.

Peripheral blood mononuclear cell (PBMC) refers to a cell with a single nucleus in peripheral blood, and includes lymphocytes, monocytes, dendritic cells and a few other cells.

Single guide RNA (sgRNA) is a single-stranded RNA with a function of crRNA-tracrRNA compound, and can be bound to a Cas9 endonuclease and guide the latter to a target site of genome for binding and cleavage.

### Embodiment 1 Preparation of Tumor Antigen PAP-GM-CSF

1. Construction of Insect Baculovirus Vector

(1) Gene Synthesis of Tumor Antigen PAP-GM-CSF

Gene sequences of prostatic acid phosphatase (PAP) and granulocyte-macrophage colony stimulating factor (GM-CSF) were retrieved from Genbank, the PAP and the GM-CSF were linked by two amino acids Gly-Ser, the PAP upstream contained a signal peptide, and codon optimization was carried out by software to synthesize a complete DNA sequence. Corresponding DNA sequence of the PAP-GM-CSF fusion protein was shown in SEQ ID NO: 1, and corresponding amino acid sequence of the PAP-GM-CSF fusion protein was shown in SEQ ID NO: 2.

(2) Construction of Shuttle Plasmid pFast-Bac1-PAP-GM-CSF

Genes of a vector pFast-Bac1 and the synthesized tumor antigen PAP-GM-CSF were enzyme digested, linked and transformed to construct a recombinant plasmid.

(3) Construction and Identification of Recombinant Bacmid PAP-GM-CSF-Bacmid

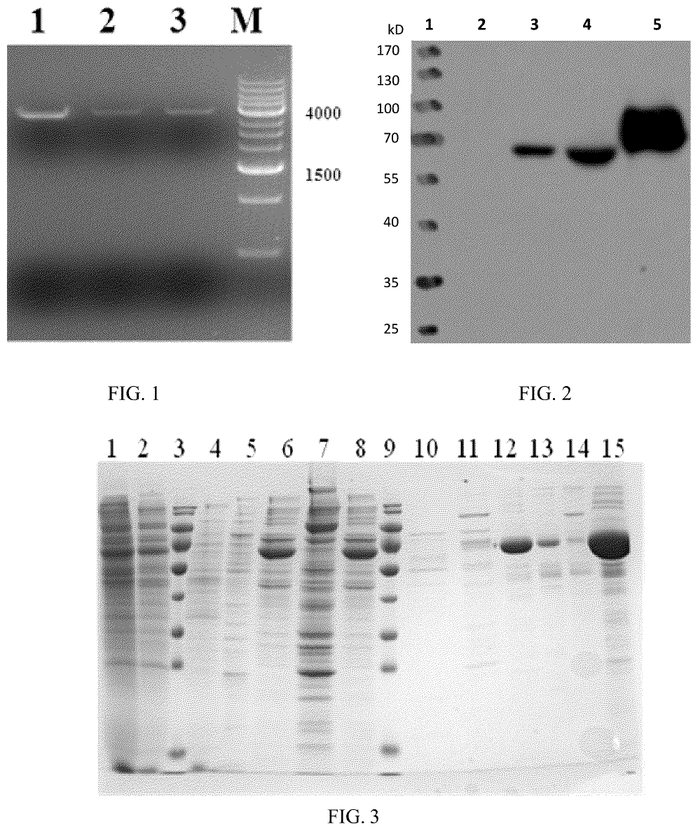

The successfully constructed shuttle plasmid pFast-Bac1-PAP-GM-CSF was extracted and transformed into an Escherichia coli competent cell DH10bac, then blue-white spot screening was carried out on a three-antibody LB plate (gentamicin, tetracycline and kanamycin), and grown white spots were purified by carrying out plate scribing (three-antibody LB plate+blue and white spot screening) for multiple times. The recombinant bacmid PAP-GM-CSF-Bacmid was extracted and used as a template, and M13F (as shown in SEQ ID NO.: 112) and M13R (as shown in SEQ ID NO.:113) were used as a primer pair for PCR amplification to verify the correctness of the recombinant bacmid. FIG. 1 shows identification results of the recombinant bacmid PAP-GM-CSF-Bacmid, wherein M represents a nucleic acid marker, and lanes 1, 2 and 3 respectively represent results of PCR amplification of three selected recombinant bacmids after bacmids extraction. PAP-GMCSF fragment size is about 1500 bp, which is increased by 2300 bp by amplification with the M13F/R primer, and the theoretical amplification band size should be close to 4000 bp, which is consistent with FIG. 1, indicating that the recombinant plasmid PAP-GM-CSF-Bacmid was successfully constructed.

2. Preparation and Identification of Recombinant Baculovirus

(1) Preparation of First (P1)-Generation Recombinant Baculovirus

On the day before transfection, insect cells Sf-9 in a logarithmic phase were spread on a 6-well plate by 1×10⁶ cells/well, and adhered overnight. A transfection process was as follows (taking transfection of one well of the 6-well plate as an example): 5 μL of recombinant bacmid PAP-GM-CSF-Bacmid was added into 100 μL of Grace's medium and mixed evenly; and 6 μL of liposome Escort™IV Transfection (SIGMA) was added into 100 μL of Grace's medium and mixed evenly. The latter was added into the former and mixed, and the mixture was incubated at a room temperature for 30 minutes. 800 μL of serum-free Grace's medium was added into each well, then a liposome-Bac mixture was added dropwise, and the mixture was incubated at 27° C. for 5 hours. The transfected mixture was removed, and 4 mL of Grace's medium (containing 5% FBS) was added, and cells were cultured in an incubator at 27° C. It could be observed under a microscope that the cells were pathologically changed on the 4^{th} day of transfection, the medium containing virus was collected and centrifuged at 1,000 g for 15 minutes, then the supernatant was transferred to a new centrifuge tube, and kept in the dark at 4° C., which was the P1-generation recombinant baculovirus.

(2) Preparation of Second (P2)-Generation Baculovirus and Third (P3)-Generation Recombinant Baculovirus

Suspended insect cells Sf-9 infected and acclimated with the collected P1-generation virus were cultured in a large scale to prepare the P2-generation baculovirus. It could be seen under a microscope that the Sf-9 cells were pathologically changed on the 3^{rd} day after being infected with the P1-generation virus, cell supernatant containing the P2-generation virus was collected, and the collected suspended insect cells Sf-9 infected and acclimated with the P2-generation virus were cultured in a large scale to prepare the P3-generation baculovirus. Meanwhile, the expression of protein in the cultural supernatant of the P2-generation baculovirus was detected by Western blotting. As shown in FIG. 2, the expression of PAP-GM-CSF in the cultural supernatant of the P2-generation recombinant baculovirus was detected by Western blotting, wherein lane 1 is PageRuler™Prestained Protein Ladder, lane 2 is the cultural supernatant of the cells Sf-9, lane 3 is the cultural supernatant of the cells Sf-9 infected with the PAP-GM/CSF baculovirus (P2-generation), lane 4 is a concentrated solution of the cultural supernatant of the cells Sf-9 infected with PAP-GM/CSF baculovirus (P2-generation), and lane 5 is a PAP-GM/CSF protein expressed by HEK293T cells. It can be seen that the size of the tumor antigen PAP-GM-CSF fusion protein of an insect cell baculovirus expression system is 64 kD, indicating that the fusion protein is the target protein.

3. Expression and Purification of Tumor Antigen PAP-GM-CSF

(1) Expression of Tumor Antigen PAP-GM-CSF Fusion Protein

The suspended insect cells Sf-9 infected and acclimated with the P3-generation recombinant baculovirus with MOI of 5 were shakily cultured in a Sf-900™II SFM serum-free insect cell medium in an incubator at 27° C. at 120 r/min 96 hours to 120 hours after infection, when the cells were pathologically changed obviously, the supernatant was collected, detected by Western blotting and purified.

(2) Purification of Tumor Antigen PAP-GM-CSF Fusion Protein

The expressed PAP-GM-CSF fusion protein was concentrated by 5 times through a Spectrumlabs hollow fiber tangential flow filtration system (with an aperture of 30 kD), and a buffer (20 mM PB, pH 7.2) was replaced.

Cation column EMD SO₃⁻ (M) flow-through: a 5×CV solution A [20 mM PB, pH 7.2] was used to balance the column bed (at a flow rate of 4 mL/min). A sample of the concentrated replaced buffer was loaded at a flow rate of 4 mL/min, the flow-through solution was collected, and then impurities were removed with a 4×CV solution B [20 mM PB, 2 M NaCl, pH 7.2].

Anion column EMD TMAE(M): a 5×CV solution A [20 mM PB, pH 7.2] was used to balance the column bed (at a flow rate of 4 mL/min). A sample of the flow-through solution collected in the first step was loaded at a flow rate of 4 mL/min. Elution was carried out with Elusion buffer 1 [20 mM PB, 100 mM NaCl, pH 7.2], Elusion buffer 2 [20 mM Pb, 200 mM NaCl, pH 7.2] and Elusion buffer 3 [20 mM PB, 2 M NaCl, pH 7.2] step by step at a flow rate of 4 mL/min, wherein an ingredient eluted with the Elution buffer 2 was the target protein.

Hydrophobic column Capto Butyl (GE): a 5×CV solution B [20 mM PB, 2 M NaCl, pH 7.2] was used to balance the column bed (at a flow rate of 2 mL/min). Before balancing the column, NaCl was carefully added into the ingredient which was eluted with the Elution buffer 2 and collected in the second step, with the concentration of NaCl reaching 2 M, and then the sample was loaded at a flow rate of 2 mL/min Elution was carried out with Elution buffer 4 [20 mM PB, 1 M NaCl, pH 7.2], Elution buffer 5 [20 mM PB, 500 mM NaCl, pH 7.2], Elution buffer 6 [20 mM PB, 200 mM NaCl, pH 7.2], and Elution buffer 7 [20 mM PB, pH 7.2] step by step at a flow rate of 2 mL/min. The ingredient eluted with Elution buffer 5 was the target protein.

Each ingredient was detected by SDS-PAGE. As shown in FIG. 3, lane 1 is a sample of PAP-GM-CSF, lane 2 is SO₃⁻ (M) flow-through, lane 3 is PageRuler™Prestained Protein Ladder, lane 4 is TMAE(M) flow-through, lane 5 is Elusion 1, lane 6 is Elusion 2, lane 7 is Elution 3, lane 8 is a pre-column sample of the Capto Butyl (GE), lane 9 is PageRuler™Prestained Protein Ladder, lane 10 is Capto Butyl (GE) flow-through, lane 11 is Elution 4, lane 12 is Elution 5, lane 13 is Elution 6, lane 14 is Elution 7, and lane 15 is Elution 5 after ultrafiltration and concentration.

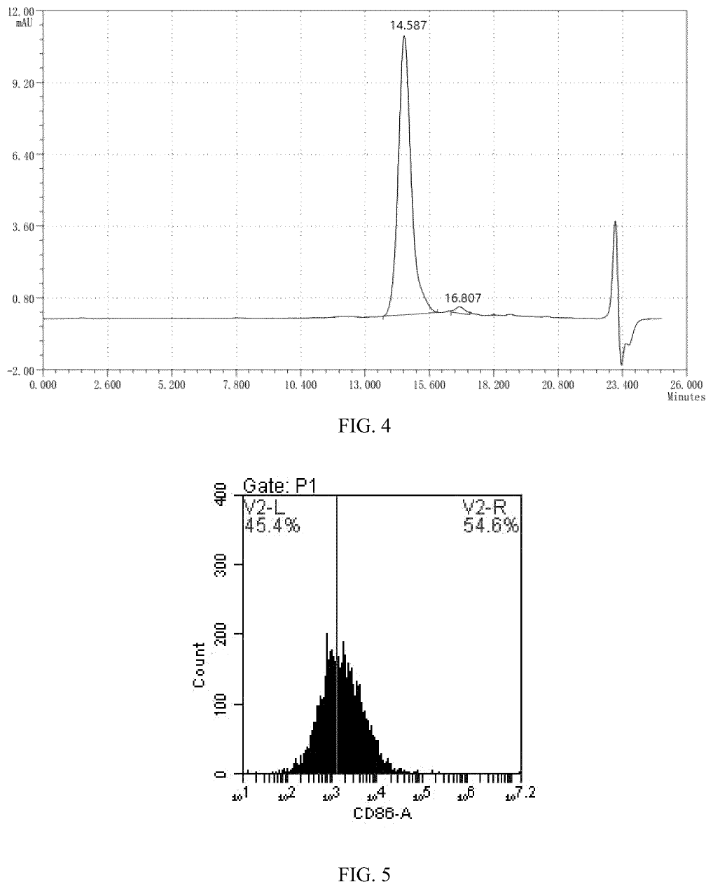

The analytical purified PAP-GM-CSF fusion protein (Elusion 5) was analyzed by HPLC. Results are shown in FIG. 4, wherein related parameters corresponding to the two ingredients are shown in Table 1. It can be seen that purity of the analytical purified PAP-GM-CSF fusion protein reaches 98%.

TABLE 1
Liquid chromatography data of analytical purified PAP-GM-CSF fusion protein

| | Retention | Peak | | | Tailing | Theoretical |
|---|---|---|---|---|---|---|
| Ingredient | time | height | Peak area | Concentration | factor | plate |
| PAP-GM-CSF | 14.587 | 10.89 | 398.131 | 98.094 | 1.43 | 3591 |
| fusion protein | | | | | | |
| Impurity | 16.807 | 0.27 | 7.734 | 1.906 | 1.14 | 7766 |
| Total | | 11.16 | 405.865 | 100.000 | | |

The ingredient eluted with the Elute buffer 5 in the third step was concentrated through the Spectrumlabs hollow fiber tangential flow filtration system (with an aperture of 30 kD), the buffer (normal saline) was replaced, filtered and sterilized, then the protein concentration was determined, and the ingredient was packaged and frozen.

(3) Immunogenicity of PAP-GM-CSF Fusion Protein

Mice were immunized with different doses of purified protein, and the antiserum titer was determined by ELISA. Reading results are shown in Table 2, it can be seen that the antiserum titer is more than 10,000. There were three experimental groups (5 mice in each grou) with immune doses being 150 μg/mouse, 100 μg/mouse and 50 μg/mouse respectively. The antibody used in positive antibody group was an Anti-GM-CSF antibody (ab54429).

TABLE 2
Results of antiserum titer of mice immunized
with different doses of PAP-GM-CSF

| Primary antibody group | | | Reading | | | | |
|---|---|---|---|---|---|---|---|
| Group | | Dilution ratio | 1 | 2 | 3 | 4 | 5 |
| Experimental groups | 150 | 10⁻¹ | 1.894 | 1.678 | 1.902 | 1.641 | 1.676 |
| (different immune | | 10⁻² | 1.884 | 1.774 | 1.885 | 1.509 | 1.681 |
| dose groups, μg/mouse) | | 10⁻³ | 1.529 | 1.446 | 1.412 | 0.798 | 1.179 |
| | | 10⁻⁴ | 0.602 | 0.569 | 0.506 | 0.309 | 0.400 |
| | 100 | 10⁻¹ | 1.764 | 1.756 | 1.762 | 1.663 | 1.781 |
| | | 10⁻² | 1.769 | 1.915 | 1.952 | 1.778 | 1.838 |
| | | 10⁻³ | 1.361 | 1.670 | 1.711 | 1.512 | 1.491 |
| | | 10⁻⁴ | 0.630 | 0.787 | 0.868 | 0.837 | 0.656 |
| | 50 | 10⁻¹ | 1.839 | 1.712 | 1.833 | 1.811 | 1.712 |
| | | 10⁻² | 1.828 | 1.847 | 1.919 | 1.879 | 1.782 |
| | | 10⁻³ | 1.524 | 1.461 | 1.514 | 1.492 | 1.561 |
| | | 10⁻⁴ | 0.554 | 0.549 | 0.577 | 0.534 | 0.606 |

| Positive antibody group | 10⁻³ | 1.250 | 1.211 | / | / | / |
|---|---|---|---|---|---|---|
| | 10⁻⁴ | 0.699 | 0.725 | / | / | / |
| Negative control group | / | 0.051 | 0.039 | 0.041 | 0.045 | / |

### Embodiment 2 DC Cells Sensitized with Tumor Antigen PAP-GM-CSF

Peripheral blood mononuclear cells (PBMC) were separated by a blood cell separator or Ficoll, CD14⁺ monocytes were separated by a magnetic bead sorting method, X-VIVO™15 (LONZA) lymphocyte serum-free medium (containing 500 U/mL to 1,000 U/mL rhGM-CSF and 500 U/mL rhIL-4) was added, then cells were cultured in a CO₂ incubator at 37° C. under 5% CO₂ The tumor antigen PAP-GM-CSF was added on the 5^{th} day to stimulate activation of DC, TNF-β (with a final concentration of 20 ng/mL) was added at the same time to induce maturation of DC, and the mixture was cultured continuously for 48 hours to obtain DC cells sensitized with the tumor antigen PAP-GM-CSF. As shown in FIG. 5, the proportion of CD86 in the DC cells is detected to be 54.6% by flow cytometry.

### Embodiment 3 Induction of CTL Cells by Using Sensitized DC Cells

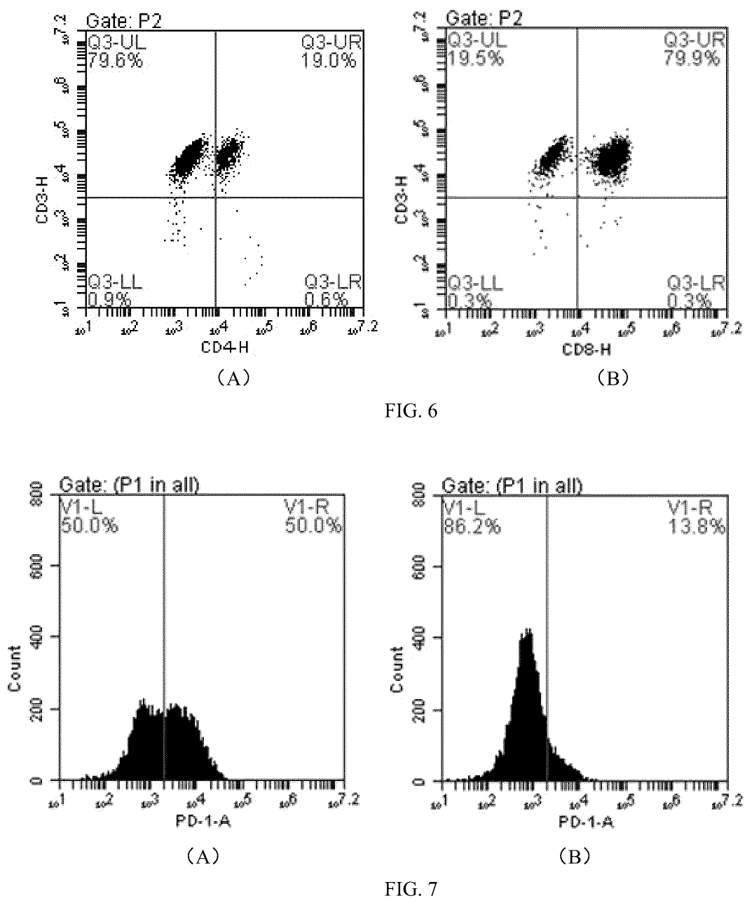

PBMCs with the same source as DCs were separated by a blood cell separator or Ficoll, and PBMCs were added into the mature DCs stimulated with the PAP-GM-CSF fusion protein in Embodiment 2 (DC: PBMC=1: 10), and co-incubated in an incubator at 37° C. under 5% CO₂ for 3 days to 5 days to obtain tumor antigen PAP-GM-CSF specific CTL cells. As shown in FIG. 6, FIG. 6(A) shows that the proportion of CD3⁺CD4⁺ in the induced cells is detected to be 19.0% by flow cytometry, and FIG. 6(B) shows that the proportion of CD3⁺CD8⁺ in the induced cells is detected to be 79.9% by flow cytometry, which means that the CTL cells reach 79.9%.

### Embodiment 4 Knockout of PD-1 Gene of Tumor Antigen-Specific CTL Cells by Crispr/Cas9 Technology

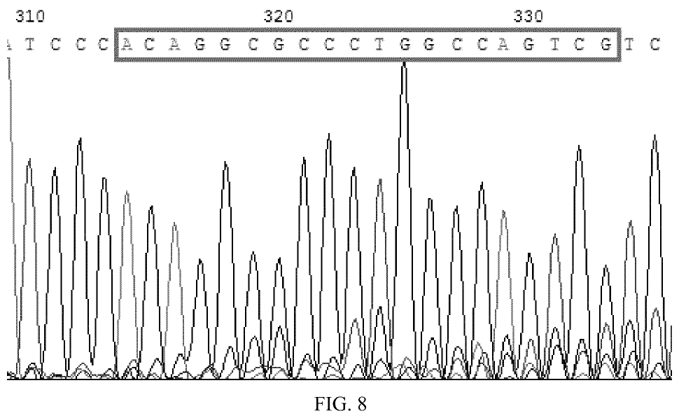

The tumor antigen PAP-GM-CSF specific CTL cells prepared in Embodiment 3 were washed with OPTI-MEM (Thermo) for three times and resuspended with OPTI-MEM, with a cell density of 5×10⁷ cells/mL. 20 μg of Cas9 protein and 10 μg of gRNA targeting the PD-1 gene transcribed in vitro were mixed evenly in advance, and incubated at a room temperature for 15 minutes. Cas9RNP and 100 μL of cell suspension were mixed evenly, then the mixture was added into a 2 mm electroporation cuvette, and electroporated by BTX ECM 830 (Harvard). The cells were quickly transferred into a 6-well plate into which 2 mL of X-VIVO™15 (LONZA) medium (containing 200 U/mL to 500 U/mL rhIL-2) preheated at 37° C. had been added, and cells were cultured in an incubator at 37° C. under 5% CO₂. On the 3R 1 day after electroporation, expression of PD-1 in the CTL cells was detected by flow cytometry. Results are shown in FIG. 7, wherein control group (A) comprises the CTL cells with the Cas9 protein electrotransformed, experimental group (B) comprises the CTL cells with the Cas9 RNP electrotransformed, and it can be seen that the expression level of PD-1 in the CTL cells is decreased from 50% to 13.8%, with a knockout efficiency reaching 72.4%. Results of PD-1 of the CTL cells verified by sanger sequencing are shown in FIG. 8, an obvious nested peak appears from the sgRNA target sequence, and it can be seen that PD-1 of the tumor antigen PAP-GM-CSF specific CTL was knocked out.

### Embodiment 5 PD-1 Knock-Out Prostate Antigen Specific CTL Cell Preparation for Treating Prostate Cancer

The electroporated cells in Embodiment 4 were cultured in a 6-well plate overnight, and then transferred into a cell culture bag to be continuously cultured for 7 days to 10 days, and a X-VIVO™15 (LONZA) medium (containing 200 U/mL to 500 U/mL rhIL-2) was added according to cell growth. Cell suspension was collected, centrifuged and washed for three times, and resuspend with 100 mL of normal saline, then 2% human serum albumin was added into the resuspension to obtain the PD-1 knock-out prostate antigen specific CTL cell preparation for treating prostate cancer, wherein PD-1 knock-out prostate antigen-specific CTL cells were more than 1×10¹⁰.

### Embodiment 6

Gene synthesis of the tumor antigen PAP-GM-CSF was the same as that in Embodiment 1.

Preparation of the tumor antigen PAP-GM-CSF: expression was carried out by HEK293T cell expression system. Results are shown in FIG. 2, wherein lane 5 is PAP-GM/CSF protein expressed by HEK293T cells, and it can be seen that the size of the PAP-GM-CSF fusion protein expressed by HEK293 cells is 75 kD, indicating that the fusion protein is the target protein. The expressed PAP-GM-CSF fusion protein was concentrated by a hollow fiber tangential flow filtration system (with an aperture of 30 kD), and purified by cation column EMD SO₃⁻ (M) flow-through, anion column EMD TMAE(M) and hydrophobic column Capto Butyl (GE).

Preparation of DC cells sensitized with the tumor antigen PAP-GM-CSF was the same as that in Embodiment 2.

Preparation of CTL cells induced by using DC cells sensitized with the tumor antigen PAP-GM-CSF was the same as that in Embodiment 3.

Knockout of PD-1 gene of the tumor antigen PAP-GM-CSF specific CTL cells was implemented by TALEN system.

### Embodiment 7

Gene synthesis of the tumor antigen PAP-GM-CSF was the same as that in Embodiment 1.

Preparation of the tumor antigen PAP-GM-CSF: expression was carried out by yeast expression system, and the expressed PAP-GM-CSF fusion protein was concentrated by a hollow fiber tangential flow filtration system (with an aperture of 30 kD), and purified by hydroxyapatite chromatography, hydrophobic chromatography and affinity chromatography.

Preparation of the DC cells sensitized with the tumor antigen PAP-GM-CSF was the same as that in Embodiment 2.

Preparation of the CTL cells induced by using the DC cells sensitized with the tumor antigen PAP-GM-CSF was the same as that in Embodiment 3.

Knockout of PD-1 gene of the tumor antigen PAP-GM-CSF specific CTL cells was implemented by a zinc finger nuclease system.

### Embodiment 8

A kit for acquiring a PD-1 knock-out CTL cell, comprising the tumor antigen PAP-GM-CSF prepared in Embodiment 1, a human peripheral blood CD14⁺ cell, a Cas9 nuclease element, a gRNA targeting a PD-1 gene, and a kit instruction.

The Kit Instruction Comprised:

Preparation of DC Cells Sensitized with Tumor Antigen PAP-GM-CS

CD14⁺ monocytes were added with an X-VIVO™15 (LONZA) lymphocyte serum-free medium (containing 500 U/mL to 1,000 U/mL rhGM-CSF and 500 U/mL rhIL-4) to culture in a CO₂ incubator at 37° C. under 5% CO₂. The tumor antigen PAP-GM-CSF was added on the 5^{th} day to stimulate activation of DC, TNF-α (with a final concentration of 20 ng/mL) was added at the same time to induce maturation of DC, and the mixture was cultured continuously for 48 hours to obtain DC cells sensitized with the tumor antigen PAP-GM-CSF.

Preparation of CTL Cell Induced by Sensitized DC Cell

PBMCs with the same source as the DCs were separated by a blood cell separator or Ficoll, and the PBMCs were added into the mature DCs stimulated with the PAP-GM-CSF fusion protein (DC: PBMC=1: 10), and co-incubated in an incubator at 37° C. under 5% CO₂ for 3 days to 5 days to obtain the tumor antigen PAP-GM-CSF specific CTL cells.

Knockout of PD-1 Gene of Tumor Antigen-Specific CTL Cell by Crispr/Cas9 Technology

The prepared PAP-GM-CSF specific CTL cells were washed with OPTI-MEM (Thermo) for three times and resuspended with OPTI-MEM, with a cell density of 5×10⁷ cells/mL. 20 μg of Cas9 protein and 10 μg of gRNA targeting PD-1 gene transcribed in vitro were mixed evenly in advance, and incubated at a room temperature for 15 minutes. Cas9RNP and 100 μL of cell suspension were mixed evenly, then the mixture were added into a 2 mm electroporation cuvette, and electroporated by BTX ECM 830 (Harvard). The cells were quickly transferred into a 6-well plate into which 2 mL of X-VIVO™15 (LONZA) medium (containing 200 U/mL to 500 U/mL rhIL-2) preheated at 37° C. had been added, and the cells were cultured in an incubator at 37° C. under 5% CO₂. The electroporated cells were cultured in the 6-well plate overnight, and then transferred into a cell culture bag to be continuously cultured for 7 days to 10 days. A X-VIVO™15 (LONZA) medium (containing 200 U/mL to 500 U/mL rhIL-2) was added according to cell growth. Then the cell suspension was collected, centrifuged, washed for three times, and resuspend with 100 mL of normal saline. 2% human serum albumin was added to obtain the PD-1 knock-out prostate antigen specific CTL cell preparation for treating prostate cancer, wherein the PD-1 knock-out prostate antigen-specific CTL cells were more than 1×10¹⁰.

It should be understood by those skilled in the art that the application of the present disclosure is not limited to the specific use above. In terms of specific elements and/or features described or depicted herein, the present disclosure is also not limited to the preferred embodiments. It should be understood that the present disclosure is not limited to the disclosed embodiments or examples, and many rearrangements, modifications and substitutions can be made without departing from the scope of the present disclosure described and defined by the following claims.

1. A preparation method for a Cytotoxic T lymphocyte, comprising the following steps of:
inducing a Cytotoxic T lymphocyte by using a DC cell sensitized with a tumor antigen PAP-GM-CSF; and
knocking out PD-1 gene of the Cytotoxic T lymphocyte to obtain a PD-1 knock-out Cytotoxic T lymphocyte.

2. The preparation method according to claim 1, wherein the tumor antigen PAP-GM-CSF is composed of PAP and GM-CSF linked by two amino acids Gly-Ser; and preferably, the PAP upstream of the tumor antigen PAP-GM-CSF comprises a signal peptide.

3. The preparation method according to claim 2, wherein the nucleotide of the tumor antigen PAP-GM-CSF is shown in SEQ ID NO.: 1; and the amino acid of the tumor antigen PAP-GM-CSF is shown in SEQ ID NO.: 2.

4. The preparation method according to claim 1, wherein the tumor antigen PAP-GM-CSF is expressed by a genetic engineering method, and the genetic engineering method is selected from one of insect cell baculovirus expression system, HEK293 cell expression system, yeast expression system and Escherichia coli expression system; preferably, the tumor antigen PAP-GM-CSF is obtained by purification after being expressed by the genetic engineering method; and the genetic engineering method is insect cell baculovirus expression system.

5. The preparation method according to claim 4, wherein the purification is implemented by ultrafiltration and continuous column chromatography.

6. The preparation method according to claim 5, wherein the continuous column chromatography is at least one selected from the group consisting of ion exchange, hydrophobic chromatography, hydroxyapatite chromatography and affinity chromatography; and preferably, the continuous column chromatography is at least one selected from the group consisting of cation column EMD SO₃⁻ (M) flow-through, anion column EMD TMAE(M) and hydrophobic column Capto Butyl.

7. The preparation method according to claim 4, wherein the purity of the tumor antigen PAP-GM-CSF is no less than 98%.

8. The preparation method according to claim 1, wherein the preparation method for the tumor antigen PAP-GM-CSF comprises the following steps of:
(1) constructing a shuttle plasmid pFast-Bac1-PAP-GM-CSF with pFast-Bac1 as a skeleton vector;
(2) transforming the shuttle plasmid into Escherichia coli, and screening to obtain a recombinant bacmid PAP-GM-CSF-Bacmid;
(3) transfecting the recombinant bacmid into an insect cell, and after the cell has an obvious pathological change, collecting the supernatant which is the first-generation baculovirus;
(4) infecting the insect cell with the first-generation baculovirus, and collecting the second-generation baculovirus or the third-generation baculovirus; and
(5) expressing PAP-GM-CSF by using a suspended insect cell infected and acclimated with the second-generation baculovirus or the third-generation baculovirus.

9. The preparation method according to claim 1, wherein the DC cell is selected from a human peripheral blood mononuclear cell, a human peripheral blood CD14⁺ cell or bone marrow.

10. The preparation method according to claim 1, wherein sensitizing the DC cell with the tumor antigen PAP-GM-CSF comprises the following steps of: adding the DC cell into a lymphocyte serum-free medium containing rhGM-CSF and rhIL-4; and after culturing, adding the tumor antigen PAP-GM-CSF and TNF-α for induction to obtain the DC cell sensitized with the tumor antigen PAP-GM-CSF.

11. The preparation method according to claim 1, wherein inducing the Cytotoxic T lymphocyte by using the DC cell sensitized with the tumor antigen PAP-GM-CSF comprises the following steps of: acquiring a human peripheral blood mononuclear cell with the same source as the DC cell, and adding the human peripheral blood mononuclear cell into the DC cell sensitized with the tumor antigen PAP-GM-CSF for co-culture to induce the Cytotoxic T lymphocyte.

12. The preparation method according to claim 1, wherein at least one selected from the group consisting of CRISPR/Cas9 system, TALEN system and zinc finger nuclease system is used to knock out PD-1 gene of the Cytotoxic T lymphocyte; and preferably, CRISPR/Cas9 system is used to knock out PD-1 gene of the Cytotoxic T lymphocyte.

13. A kit for acquiring a Cytotoxic T lymphocyte, wherein the kit comprises the tumor antigen PAP-GM-CSF according to claim 1; and preferably, the kit further comprises a Cas9 nuclease element, a gRNA targeting PD-1 gene, and a kit instruction, and the preparation method according to claim 1 is recorded in the kit instruction.

14. A method of treating PAP-positive prostate cancer, comprising administering a therapeutically effective amount of Cytotoxic T lymphocytes according to claim 1 to a subject in need thereof.

15. The preparation method according to claim 5, wherein the purity of the tumor antigen PAP-GM-CSF is no less than 98%.

16. The preparation method according to claim 6, wherein the purity of the tumor antigen PAP-GM-CSF is no less than 98%.


SEQUENCE LISTING (113 sequences)

SEQ ID NO: 1 - DNA, 1548 bp, Artificial Sequence
  synthesized
atgcgtgccgctcccctgctgctggcccgtgctgcttccctgtccctgggtttcctgttcctgctgttcttctggctggaccgcagcgtgctggctaaggagctgaagttcgtgaccctggtgttccgccacggagatcgcagtcccatcgataccttccccaccgatcccatcaaggagagcagttggccacagggattcggacaactgacccaactgggcatggagcagcactacgagctgggagagtacatccgcaagcgctaccgcaagttcctgaacgagagctacaagcacgagcaggtgtacatccgctcgaccgatgtggatcgcaccctgatgagcgctatgaccaacctggccgctctgttcccaccagagggcgtgtccatttggaaccccatcctgctgtggcagccaatcccagtgcacacagtgccactgagcgaggatcaactgctgtacctgcccttccgcaattgcccacgtttccaagagttggagagcgagaccctgaagagcgaggagttccaaaagcgcctgcacccctacaaggacttcatcgccaccctgggaaagctgagcggattgcacggccaggacctgttcggcatttggagcaaggtgtacgaccccctgtattgcgagagcgtgcacaacttcaccctgcccagttgggctaccgaggataccatgaccaagctgcgcgagctgagcgaactgagtctgctgagcctgtacggcatccacaagcagaaggagaagagccgtctgcaaggcggagtgctggtgaacgagatcctgaaccacatgaagcgtgccacccagatccccagctacaagaagctgatcatgtacagcgcccacgataccacagtgtccggactgcaaatggccctggacgtgtacaacggcctgttgccaccatacgccagttgccacctgaccgagctgtacttcgagaagggcgagtacttcgtggagatgtactaccgcaacgagacccagcacgagccatacccactgatgctgccaggctgctccccaagttgcccactggaacgcttcgccgaattggtgggaccagtgatcccacaggattggagcaccgagtgcatgaccaccaacagccaccagggaaccgaggattcgaccgatggtagcgctccagctcgtagcccaagcccaagtactcagccctgggagcacgtgaacgctatccaggaagcccgtcgcctgctgaatctgagtcgcgatacagccgccgagatgaacgagaccgtggaggtcatcagcgagatgttcgacctgcaagagcccacttgcctgcaaacacgtctggagctgtacaagcagggactgcgaggaagcctgaccaagctgaagggaccactgaccatgatggccagccactacaagcagcattgcccaccaacaccagagacaagttgcgccacccagatcatcaccttcgagagcttcaaggagaacctgaaggacttcctgctggtcatccccttcgattgttgggagccagtgcaggagtaa

SEQ ID NO: 2 - protein, 483 aa, Artificial Sequence
  synthesized
KELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTDGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 3 - DNA, 20 bp, Artificial Sequence
  synthesized
cgactggccagggcgcctgt

SEQ ID NO: 4 - DNA, 20 bp, Artificial Sequence
  synthesized
cgtctgggcggtgctacaac

SEQ ID NO: 5 - DNA, 20 bp, Artificial Sequence
  synthesized
tgtagcaccgcccagacgac

SEQ ID NO: 6 - DNA, 20 bp, Artificial Sequence
  synthesized
accgcccagacgactggcca

SEQ ID NO: 7 - DNA, 20 bp, Artificial Sequence
  synthesized
aggcgccctggccagtcgtc

SEQ ID NO: 8 - DNA, 20 bp, Artificial Sequence
  synthesized
gtctgggcggtgctacaact

SEQ ID NO: 9 - DNA, 20 bp, Artificial Sequence
  synthesized
ggcgccctggccagtcgtct

SEQ ID NO: 10 - DNA, 20 bp, Artificial Sequence
  synthesized
caccgcccagacgactggcc

SEQ ID NO: 11 - DNA, 20 bp, Artificial Sequence
  synthesized
gggcggtgctacaactgggc

SEQ ID NO: 12 - DNA, 20 bp, Artificial Sequence
  synthesized
gccctggccagtcgtctggg

SEQ ID NO: 13 - DNA, 20 bp, Artificial Sequence
  synthesized
ctacaactgggctggcggcc

SEQ ID NO: 14 - DNA, 20 bp, Artificial Sequence
  synthesized
acgactggccagggcgcctg

SEQ ID NO: 15 - DNA, 20 bp, Artificial Sequence
  synthesized
cggtgctacaactgggctgg

SEQ ID NO: 16 - DNA, 20 bp, Artificial Sequence
  synthesized
tgcagatcccacaggcgccc

SEQ ID NO: 17 - DNA, 20 bp, Artificial Sequence
  synthesized
tccaggcatgcagatcccac

SEQ ID NO: 18 - DNA, 20 bp, Artificial Sequence
  synthesized
gcctgtgggatctgcatgcc

SEQ ID NO: 19 - DNA, 20 bp, Artificial Sequence
  synthesized
aactgggctggcggccagga

SEQ ID NO: 20 - DNA, 20 bp, Artificial Sequence
  synthesized
ggccaggatggttcttaggt

SEQ ID NO: 21 - DNA, 20 bp, Artificial Sequence
  synthesized
tggcggccaggatggttctt

SEQ ID NO: 22 - DNA, 20 bp, Artificial Sequence
  synthesized
atgtggaagtcacgcccgtt

SEQ ID NO: 23 - DNA, 20 bp, Artificial Sequence
  synthesized
cacgaagctctccgatgtgt

SEQ ID NO: 24 - DNA, 20 bp, Artificial Sequence
  synthesized
cctgctcgtggtgaccgaag

SEQ ID NO: 25 - DNA, 20 bp, Artificial Sequence
  synthesized
catgtggaagtcacgcccgt

SEQ ID NO: 26 - DNA, 20 bp, Artificial Sequence
  synthesized
cccttcggtcaccacgagca

SEQ ID NO: 27 - DNA, 20 bp, Artificial Sequence
  synthesized
cggagagcttcgtgctaaac

SEQ ID NO: 28 - DNA, 20 bp, Artificial Sequence
  synthesized
gcgtgacttccacatgagcg

SEQ ID NO: 29 - DNA, 20 bp, Artificial Sequence
  synthesized
gccctgctcgtggtgaccga

SEQ ID NO: 30 - DNA, 20 bp, Artificial Sequence
  synthesized
aggcggccagcttgtccgtc

SEQ ID NO: 31 - DNA, 20 bp, Artificial Sequence
  synthesized
acttccacatgagcgtggtc

SEQ ID NO: 32 - DNA, 20 bp, Artificial Sequence
  synthesized
ggtgccgctgtcattgcgcc

SEQ ID NO: 33 - DNA, 20 bp, Artificial Sequence
  synthesized
ccccttcggtcaccacgagc

SEQ ID NO: 34 - DNA, 20 bp, Artificial Sequence
  synthesized
ccctgctcgtggtgaccgaa

SEQ ID NO: 35 - DNA, 20 bp, Artificial Sequence
  synthesized
tctctttgatctgcgccttg

SEQ ID NO: 36 - DNA, 20 bp, Artificial Sequence
  synthesized
tgacacggaagcggcagtcc

SEQ ID NO: 37 - DNA, 20 bp, Artificial Sequence
  synthesized
aggtgccgctgtcattgcgc

SEQ ID NO: 38 - DNA, 20 bp, Artificial Sequence
  synthesized
gctctctttgatctgcgcct

SEQ ID NO: 39 - DNA, 20 bp, Artificial Sequence
  synthesized
ctctctttgatctgcgcctt

SEQ ID NO: 40 - DNA, 20 bp, Artificial Sequence
  synthesized
agcttgtccgtctggttgct

SEQ ID NO: 41 - DNA, 20 bp, Artificial Sequence
  synthesized
agggcccggcgcaatgacag

SEQ ID NO: 42 - DNA, 20 bp, Artificial Sequence
  synthesized
cagcttgtccgtctggttgc

SEQ ID NO: 43 - DNA, 20 bp, Artificial Sequence
  synthesized
gggccctgaccacgctcatg

SEQ ID NO: 44 - DNA, 20 bp, Artificial Sequence
  synthesized
ctctttgatctgcgccttgg

SEQ ID NO: 45 - DNA, 20 bp, Artificial Sequence
  synthesized
cttccacatgagcgtggtca

SEQ ID NO: 46 - DNA, 20 bp, Artificial Sequence
  synthesized
gcagttgtgtgacacggaag

SEQ ID NO: 47 - DNA, 20 bp, Artificial Sequence
  synthesized
cagcaaccagacggacaagc

SEQ ID NO: 48 - DNA, 20 bp, Artificial Sequence
  synthesized
gtgtcacacaactgcccaac

SEQ ID NO: 49 - DNA, 20 bp, Artificial Sequence
  synthesized
gcttgtccgtctggttgctg

SEQ ID NO: 50 - DNA, 20 bp, Artificial Sequence
  synthesized
cgttgggcagttgtgtgaca

SEQ ID NO: 51 - DNA, 20 bp, Artificial Sequence
  synthesized
acagcggcacctacctctgt

SEQ ID NO: 52 - DNA, 20 bp, Artificial Sequence
  synthesized
tcggtcaccacgagcagggc

SEQ ID NO: 53 - DNA, 20 bp, Artificial Sequence
  synthesized
cgggctggctgcggtcctcg

SEQ ID NO: 54 - DNA, 20 bp, Artificial Sequence
  synthesized
ccgggctggctgcggtcctc

SEQ ID NO: 55 - DNA, 20 bp, Artificial Sequence
  synthesized
gaaggtggcgttgtcccctt

SEQ ID NO: 56 - DNA, 20 bp, Artificial Sequence
  synthesized
gacagcggcacctacctctg

SEQ ID NO: 57 - DNA, 20 bp, Artificial Sequence
  synthesized
ctggctgcggtcctcgggga

SEQ ID NO: 58 - DNA, 20 bp, Artificial Sequence
  synthesized
caagctggccgccttccccg

SEQ ID NO: 59 - DNA, 20 bp, Artificial Sequence
  synthesized
cgtgtcacacaactgcccaa

SEQ ID NO: 60 - DNA, 20 bp, Artificial Sequence
  synthesized
acggaagcggcagtcctggc

SEQ ID NO: 61 - DNA, 20 bp, Artificial Sequence
  synthesized
gatctgcgccttgggggcca

SEQ ID NO: 62 - DNA, 20 bp, Artificial Sequence
  synthesized
tgatctgcgccttgggggcc

SEQ ID NO: 63 - DNA, 20 bp, Artificial Sequence
  synthesized
acatgagcgtggtcagggcc

SEQ ID NO: 64 - DNA, 20 bp, Artificial Sequence
  synthesized
cagcggcacctacctctgtg

SEQ ID NO: 65 - DNA, 20 bp, Artificial Sequence
  synthesized
gccgggctggctgcggtcct

SEQ ID NO: 66 - DNA, 20 bp, Artificial Sequence
  synthesized
cgcagatcaaagagagcctg

SEQ ID NO: 67 - DNA, 20 bp, Artificial Sequence
  synthesized
ctgcagcttctccaacacat

SEQ ID NO: 68 - DNA, 20 bp, Artificial Sequence
  synthesized
cggaagcggcagtcctggcc

SEQ ID NO: 69 - DNA, 20 bp, Artificial Sequence
  synthesized
cggtcaccacgagcagggct

SEQ ID NO: 70 - DNA, 20 bp, Artificial Sequence
  synthesized
agcggcagtcctggccgggc

SEQ ID NO: 71 - DNA, 20 bp, Artificial Sequence
  synthesized
catgagccccagcaaccaga

SEQ ID NO: 72 - DNA, 20 bp, Artificial Sequence
  synthesized
gcagatcaaagagagcctgc

SEQ ID NO: 73 - DNA, 20 bp, Artificial Sequence
  synthesized
cctctgtggggccatctccc

SEQ ID NO: 74 - DNA, 20 bp, Artificial Sequence
  synthesized
tcaccctgagctctgcccgc

SEQ ID NO: 75 - DNA, 20 bp, Artificial Sequence
  synthesized
tctggttgctggggctcatg

SEQ ID NO: 76 - DNA, 20 bp, Artificial Sequence
  synthesized
gctgcggtcctcggggaagg

SEQ ID NO: 77 - DNA, 20 bp, Artificial Sequence
  synthesized
cgatgtgttggagaagctgc

SEQ ID NO: 78 - DNA, 20 bp, Artificial Sequence
  synthesized
gagaaggtgggggggttcca

SEQ ID NO: 79 - DNA, 20 bp, Artificial Sequence
  synthesized
ccagggagatggccccacag

SEQ ID NO: 80 - DNA, 20 bp, Artificial Sequence
  synthesized
gggggttccagggcctgtct

SEQ ID NO: 81 - DNA, 20 bp, Artificial Sequence
  synthesized
ggagatggccccacagaggt

SEQ ID NO: 82 - DNA, 20 bp, Artificial Sequence
  synthesized
ggtcaccacgagcagggctg

SEQ ID NO: 83 - DNA, 20 bp, Artificial Sequence
  synthesized
agagcctgcgggcagagctc

SEQ ID NO: 84 - DNA, 20 bp, Artificial Sequence
  synthesized
ggagaaggtgggggggttcc

SEQ ID NO: 85 - DNA, 20 bp, Artificial Sequence
  synthesized
gagcctgcgggcagagctca

SEQ ID NO: 86 - DNA, 20 bp, Artificial Sequence
  synthesized
ggggggttccagggcctgtc

SEQ ID NO: 87 - DNA, 20 bp, Artificial Sequence
  synthesized
cttctccccagccctgctcg

SEQ ID NO: 88 - DNA, 20 bp, Artificial Sequence
  synthesized
cccgaggaccgcagccagcc

SEQ ID NO: 89 - DNA, 20 bp, Artificial Sequence
  synthesized
ggccatctccctggccccca

SEQ ID NO: 90 - DNA, 20 bp, Artificial Sequence
  synthesized
ggaccgcagccagcccggcc

SEQ ID NO: 91 - DNA, 20 bp, Artificial Sequence
  synthesized
ggggttccagggcctgtctg

SEQ ID NO: 92 - DNA, 20 bp, Artificial Sequence
  synthesized
cgccttgggggccagggaga

SEQ ID NO: 93 - DNA, 20 bp, Artificial Sequence
  synthesized
gttggagaagctgcaggtga

SEQ ID NO: 94 - DNA, 20 bp, Artificial Sequence
  synthesized
atctctcagactccccagac

SEQ ID NO: 95 - DNA, 20 bp, Artificial Sequence
  synthesized
agtcctggccgggctggctg

SEQ ID NO: 96 - DNA, 20 bp, Artificial Sequence
  synthesized
cgggcagagctcagggtgac

SEQ ID NO: 97 - DNA, 20 bp, Artificial Sequence
  synthesized
agagctcagggtgacaggtg

SEQ ID NO: 98 - DNA, 20 bp, Artificial Sequence
  synthesized
tgtctggggagtctgagaga

SEQ ID NO: 99 - DNA, 20 bp, Artificial Sequence
  synthesized
cacgagcagggctggggaga

SEQ ID NO: 100 - DNA, 20 bp, Artificial Sequence
  synthesized
cagactccccagacaggccc

SEQ ID NO: 101 - DNA, 20 bp, Artificial Sequence
  synthesized
ggagaagctgcaggtgaagg

SEQ ID NO: 102 - DNA, 20 bp, Artificial Sequence
  synthesized
agcagggctggggagaaggt

SEQ ID NO: 103 - DNA, 20 bp, Artificial Sequence
  synthesized
gcagggctggggagaaggtg

SEQ ID NO: 104 - DNA, 20 bp, Artificial Sequence
  synthesized
cagggctggggagaaggtgg

SEQ ID NO: 105 - DNA, 20 bp, Artificial Sequence
  synthesized
agggctggggagaaggtggg

SEQ ID NO: 106 - DNA, 20 bp, Artificial Sequence
  synthesized
gagcagggctggggagaagg

SEQ ID NO: 107 - DNA, 20 bp, Artificial Sequence
  synthesized
gggctggggagaaggtgggg

SEQ ID NO: 108 - DNA, 20 bp, Artificial Sequence
  synthesized
cacgaagctctccgatgtgt

SEQ ID NO: 109 - DNA, 20 bp, Artificial Sequence
  synthesized
cccttcggtcaccacgagca

SEQ ID NO: 110 - DNA, 20 bp, Artificial Sequence
  synthesized
cctgctcgtggtgaccgaag

SEQ ID NO: 111 - DNA, 82 bp, Artificial Sequence
  synthesized
gttttagagctagaaatagcaagttaaaataaggctagtccgttatcaacttgaaaaagtggcaccgagtcggtgctttttt

SEQ ID NO: 112 - DNA, 17 bp, Artificial Sequence
  synthesized
gttttcccagtcacgac

SEQ ID NO: 113 - DNA, 17 bp, Artificial Sequence
  synthesized
caggaaacagctatgac